(19) United States
(12) Patent Application Publication
(10) Pub. No.: US 2004/0002108 A1
(43) Pub. Date: Jan. 1, 2004

(54) Predicting the significance of single nucleotide polymorphisms (SNPs) using ensemble-based structural energetics

(76) Inventors: Vince Hilser, Galveston, TX (US); Robert O. Fox, Galveston, TX (US)

Correspondence Address: FULBRIGHT & JAWORSKI, LLP, 1301 MCKINNEY, SUITE 5100, HOUSTON, TX, 77010-3095, US

(21) Appl. No.: 10/440,701

(22) Filed: May 19, 2003

Related U.S. Application Data
(60) Provisional application No. 60/382,784, filed on May 23, 2002.

(51) Int. Cl. C12Q 1/68; G01N 33/53; G06F 19/00; G01N 33/48; G01N 33/50

(52) U.S. Cl. 435/6; 435/7.1; 702/19

(57) ABSTRACT

The present invention relates to a protein database and methods of developing a protein database that contains identified single nucleotide polymorphisms that are relevant to protein function. Yet further, the present invention relates to utilizing the protein database to design protein pharmaceuticals that have optimized pharmaceutical properties.

This application claims priority to U.S. Provisional Application No. 60/382,784 filed May 23, 2002, which is incorporated herein by reference.

The work herein was supported by grants from the United States Government. The United States Government may have certain rights in the invention.

### FIELD OF THE INVENTION

The present invention relates to the field of structural biology. More particularly, the present invention relates to a protein database and methods of developing a protein database that contains identified single nucleotide polymorphisms that are relevant to protein function. Yet further, the present invention relates to utilizing the protein database to design protein pharmaceuticals that have optimized pharmaceutical properties.

### BACKGROUND OF THE INVENTION

Natural DNA sequence variation exists in identical genomic regions of DNA among individual members of a species. It is of interest to identify similarities and differences in such genomic regions of DNA because such information can help identify sequences involved in susceptibility to disease states as well as provide genetic information for characterization and analysis of genetic material.

When a cell undergoes reproduction, its DNA molecules are replicated and precise copies are passed on to its descendants. The linear base sequence of a DNA molecule is maintained during replication by complementary DNA base pairing. Occasionally, an incorrect base pairing does occur during DNA replication, which, after further replication of the new strand, results in a double-stranded DNA offspring with a sequence containing a heritable single base difference from that of the parent DNA molecule. Such heritable changes are called “genetic polymorphisms,” “genetic mutations,” “single base pair mutations,” “point mutations” or simply, “DNA mismatches”. In addition to random mutations during DNA replication, organisms are constantly bombarded by endogenous and exogenous genotoxic agents, which injure or damage DNA. Such DNA damage or injury can result in the formation of DNA mismatches or DNA mutations such as insertions or deletions.

The consequences of natural DNA sequence variation, DNA mutations, DNA mismatches and DNA damage range from negligible to lethal, depending on the location and effect of the sequence change in relation to the genetic information encoded by the DNA. In some instances, natural DNA sequence variation, DNA mutations, DNA mismatches and DNA damage can lead to cancer and other diseases of which early detection is critical for treatment.

There is, thus, a tremendous need to be able to rapidly identify differences in DNA sequences among individuals. In addition, there is a need to identify DNA mutations, DNA mismatches and DNA damage to provide for early detection of cancer and other.

Currently, bioinformatic algorithms attempt to correlate single nucleotide polymorphism (SNPs) with disease states by inputting the entire genome of the individuals into a database to determine if they are genotypically positive for the disease. This process is complicated by the fact that the SNP or SNPs that are responsible for a given disease are in a background of inconsequential differences. This background “noise” is one of the reasons why such approaches are unable to uniquely identify the SNP responsible for a particular disease.

The present invention is the first to use a set of algorithms that identify functionally relevant SNPs without identifying the “noise” from the signals which leads to an increase in efficiency of the above approaches.

### BRIEF SUMMARY OF THE INVENTION

The present invention is directed to a database and methods for developing a database that contains identified single nucleotide polymorphisms that are relevant to the function of the protein. This database can be utilized to design protein pharmaceuticals with optimized pharmaceutical properties, such as binding affinity.

An embodiment of the present invention is a protein database comprising nonhomologus proteins having identified single nucleotide polymorphisms that are relevant to the function of the protein, for example binding of the protein.

The database is determined by a computational method comprising the step of determining the residue-specific connectivity between residues in the protein according to the equation

$$RSC ( j , k ) = \frac{⟨ ( {S}_{j} - {\overline{S}}_{j} ) \cdot ( {S}_{k} - {\overline{S}}_{k} ) ⟩}{{( ⟨ {( {S}_{j} - {\overline{S}}_{j} )}^{2} ⟩ \cdot ⟨ {( {S}_{k} - {\overline{S}}_{k} )}^{2} ⟩ )}^{1 / {2}^{′}}}$$

Still further, the database is determined by a computational method comprising the step of determining the functional connectivity between the binding site of the protein and every residue in the protein according to the equation FCₓ(j*, k*)=RSC(j, k).

Another embodiment of the present invention is a method of predicting a subset of the population at risk for an adverse side effect of a pharmaceutical composition comprising using the database of the present invention to determine the subset having a relevant single nucleotide polymorphism interfering with an active site of a protein.

Still yet, another embodiment is a method of targeting a pharmaceutical composition for an active site of a protein comprising using the database of the present invention to determine the interactions of single nucleotide polymorphisms on the active site of a protein and designing the pharmaceutical composition to overcome the interactions of the single nucleotide polymorphism so that the pharmaceutical composition binds to the active site of the protein.

In another embodiment, the present invention provides a method of developing a protein database comprising the steps of: inputting high resolution structures of proteins; generating an ensemble of incrementally different conformational states by combinatorial unfolding of a set of predefined folding units in all possible combinations of each protein; determining the probability of each said conformational state; calculating a residue-specific connectivity of each said conformational state; and calculating a functional connectivity of each of said conformation state.

A specific embodiment is a system for developing a protein database having identified single nucleotide polymorphisms that are relevant to the function of the protein comprising a protein database having a data structure for protein data, said data structure including data fields for relevant single nucleotide polymorphisms; and a computer-based program for identifying protein data for said database, said program having an input module for receiving high resolution structure data for one or more proteins, and a processing module for determining the relevance of single nucleotide polymorphisms in the active site of one or more proteins and storing said data into said data fields of said protein database. The computer program further includes a display module for producing one or more graphical reports to a screen or a print-out.

Another embodiment of the present invention includes a database having a data structure which stores information defining relevant single nucleotide polymorphism groups, said database comprising: a field for storing a value of an amino acid name or amino acid abbreviation; and one or more classification fields for storing a value representing a numerical value for a relevant single nucleotide polymorphism.

Still further, another embodiment is a method of designing a protein pharmaceutical exhibiting optimized pharmaceutical properties comprising the steps of: obtaining a test data set of variants of the protein pharmaceutical, wherein the variants comprise single nucleotide polymorphisms; preparing a library of ensemble derived properties for the test data set using a computer based method; obtaining experimental data for a given property for each protein variant within the test data set; deriving a parametric equation using the experimental data and the library of ensemble derived properties; and creating a protein pharmaceutical using the information obtained by the above steps to provide optimized pharmaceutical properties. The optimized property is increased binding affinity.

Another embodiment of the present invention is a method of identifying relevant single nucleotide polymorphisms comprising the steps of: inputting high resolution structures of proteins; generating an ensemble of incrementally different conformational states by combinatorial unfolding of a set of predefined folding units in all possible combinations of each protein; determining the probability of each said conformational state; calculating a residue-specific connectivity of each said conformational state; and calculating a functional connectivity of each of said conformation state.

The foregoing has outlined rather broadly the features and technical advantages of the present invention in order that the detailed description of the invention that follows may be better understood. Additional features and advantages of the invention will be described hereinafter which form the subject of the claims of the invention. It should be appreciated by those skilled in the art that the conception and specific embodiment disclosed may be readily utilized as a basis for modifying or designing other structures for carrying out the same purposes of the present invention. It should also be realized by those skilled in the art that such equivalent constructions do not depart from the spirit and scope of the invention as set forth in the appended claims. The novel features which are believed to be characteristic of the invention, both as to its organization and method of operation, together with further objects and advantages will be better understood from the following description when considered in connection with the accompanying figures. It is to be expressly understood, however, that each of the figures is provided for the purpose of illustration and description only and is not intended as a definition of the limits of the present invention.

### BRIEF DESCRIPTION OF THE DRAWINGS

For a more complete understanding of the present invention, reference is now made to the following descriptions taken in conjunction with the accompanying drawings.

FIG. 1 is a flow chart the MPMOD program.

FIG. 2 is a flow chart of BEST/MPMOD program as an analysis tool.

FIG. 3A AND FIG. 3B are a flow chart of BEST/MPMOD program as a predictive tool to be used in the development of protein pharmaceuticals.

FIG. 4A and FIG. 4B show a stability plot and a ribbon plot of CDK41, respectively. FIG. 4A shows a stability plot of CDK4I calculated using COREX. Non-synonymous single nucleotide substitutions (missense) that results in aberrant phenotype are indicated in filled triangles and nonsense mutations are indicated by filled squares below the line. Most of SNPs that result in diseased state are clustered in the high stability region indicated by a downward arrow. Open boxes on the line are the residues that make contact with cyclin-dependent kinase 4 and 6. FIG. 4B shows a ribbon plot of CDK4I experimental structure (PDB code: 1B17). Point mutations that result in aberrant phenotype are shown in dark gray.

### DETAILED DESCRIPTION OF THE INVENTION

The present invention uses the COREX algorithm to generate a large number of partially folded states of a protein from the high resolution crystallographic or NMR structure (Hilser & Freire, 1996; Hilser & Freire, 1997; Hilser et al., 1997). COREX is used to calculate the residue-specific and functional connectivities in proteins (Pan et al., 2000). The determination of the residue-specific and functional connectivities determine how changes at one region of the protein are propagated to other regions. For the purpose of identifying SNPs, the ability to map the effects of changes at each residue on all other residues provides a means of cataloguing those genetic variations that are likely to affect the active site or regulatory sites on a specific protein known to be associated with a genetic disease.

I. Definitions

“A” or “an”, as used herein the specification, may mean one or more. As used herein in the claim(s), when used in conjunction with the word “comprising”, the words “a” or “an” may mean one or more than one.

Another, as used herein, may mean at least a second or more.

Autologous protein, polypeptide or peptide, as used herein, refers to a protein, polypeptide or peptide which is derived or obtained from an organism.

Based upon a tertiary structure, as used herein, refers to a structure that possesses a similar backbone structure to that of the original structure that it is referred to being based upon.

Configuration, as used herein, refers to different conformations of a protein molecule that have the same chirality of atoms.

Conformation or conformer, as used herein, refers to various nonsuperimposable three-dimensional arrangements of atoms that are interconvertible without breaking covalent bonds.

Computer modeling, as used herein, refers to the construction of patterns using raw data to simulate an object or the interaction of objects using a computer. For example, computer modeling is used to determine the size, shape, and interaction of certain compounds in order to develop treatments associated to a specific disease.

Computer simulation, as used herein, refers to a software program that runs on any size computer that attempts to simulate some phenomenon based on a scientist's conceptual and mathematical understanding of the phenomenon. The scientist's conceptual understanding is reduced to an algorithmic or mathematical logic, which is then programmed in one of many programming languages and compiled to produce a binary code that runs on a computer. Also, the act of running such a code on a computer.

Constrained, as used herein, refers to a limitation in the conformational space that the peptide may adopt.

Database, as used herein, refers to any compilation of information regarding the relation of experimental and analytical data of a protein. The database used may be publicly available, commercially available or one created by the inventors. Thus, a database is a collection of data arranged for ease of retrieval by a computer. Data are stored in a manner where it is easily compared to existing data sets.

Disulfide bridge or disulflde bond, as used herein, refers to a covalent bond between the sulfur atoms of two cysteines.

Generate or generating, as used herein, refers the act of defining or originating by the use of one or more operations. Skilled artisans using the invention may create the matter or data themselves or locate the matter or data elsewhere and utilize it in the practice of the invention. One skilled in the art realizes that in this invention all of the test data or experimental data may be obtained commercially or publicly or generated by procedures and techniques defined herein. The terms “generating” and “obtaining” are mutually inclusive as used herein.

Homologous protein, polypeptide or peptide, as used herein, refers to a protein, polypeptide or peptide which is derived or obtained from a similar organism or an organism with a common ancestry.

Ligand, as used herein, refers to a proteinaceous or non-proteinaceous compound. The ligand may be, but is not limited to, a receptor, an enzyme, a coenzyme, or a non-proteinaceous chemical compound.

Loop, as used herein, are turns in the polypeptide chain that reverse the direction of the polypeptide chain at the surface of the molecule.

Mutation(s), as used herein, refers to a change of one or more amino acids in a protein.

Nonhomologous protein, polypeptide or peptide, as used herein, refers to a protein, polypeptide or peptide which is derived or obtained from another organism that is not similar or an organism without a common ancestry.

Parametric equation, as used herein, refers to an equation containing variables related to the populated conformational states for a given variant. The equation utilizes the experimental data acquired for each variant and the library of ensemble-derived properties.

Peptide, as used herein, refers to a chain of amino acids with a defined sequence whose physical properties are those expected from the sum of its amino acid residues and there is no fixed three-dimensional structure).

Pharmaceutical properties, as used herein, refer to, but are not limited to, binding affinity, aggregation, solubility, and immunogenic effects.

Protein, as used herein, refers to a chain of amino acid residues usually of defined sequence, length and three dimensional structure. The polymerization reaction which produces a protein results in the loss of one molecule of water from each amino acid. Proteins are often said to be composed of amino acid residues. Natural protein molecules may contain as many as 20 different types of amino acid residues, each of which contains a distinctive side chain. A protein may be composed of multiple peptides.

Protein function, as used herein, refers to the contribution and/or action of the protein. For example, protein function includes protein binding (e.g., binding of the protein to another protein, nucleic acid, or small molecule to result in metabolic regulation; signal transduction; gene regulation, etc.) Protein function may also include the known biological roles of proteins, for example, enzymatic (i.e., pepsin), transport (i.e, hemoglobin), structural (i.e., collagen), storage (i.e., casein), hormonal (i.e., insulin), receptor, contractile (i.e., actin) and defensive (i.e., antibody).

Rotamer, as used herein, refers to a low energy amino acid side chain information.

Single nucleotide polymorphisms or SNP or SNPs, as used herein, refers to common DNA sequence variations among individuals. The DNA sequence variation is typically a single base change or point mutation resulting in genetic variation between individuals.

Solubility, as used herein, refers to the amount of the protein that can be dissolved in a given volume of a solvent.

Structural Characteristics, as used herein, refers to the characteristics that are determined using the computer-assisted program, such as, but not limited to folding characteristics, disulfide bonding, binding affinity, aggregation, solubility, immunogenicity, stability, etc. Thus, one of skill in the art realizes that the present invention is used to determine any structural characteristic of a protein and this characteristic may be enhanced or reduced depending upon the application of use.

Template molecule, as used herein, refers to the protein to which the modified protein is binding.

Variant, as used herein, refers to a protein with a given set of mutation(s), i.e., SNPs.

II. COREX Modeling Strategy

As opposed to modeling proteins as rigid bodies with local side-chain and backbone motions, the COREX algorithm models the native state of a protein as an ensemble of >10⁵ conformational states. The power of the ensemble-based approach is that it models states in a combined fashion: For a given state, regions that are folded are modeled according to the high-resolution structure, while regions that are unfolded are modeled as having a conformational entropy (as opposed to being modeled as a large number of specific microscopic conformations). As the entropy change (ΔS) is related to the difference in the number of conformational states (i.e., ΔS=R1nΩ, where Ω is the ratio of the degrees of freedom (or number of microscopic states) in the particular macroscopic state relative to the degrees of freedom in the reference or fully folded state), a large number of microscopic states can be represented by a single macroscopic state. The implication of this dual modeling procedure is that a macroscopic state that has 10 residues unfolded (with a minimum of 4 conformations per residue) would require modeling over 1 million explicit microscopic conformational states. The end result of the calculation is that with 100,000 macroscopic states in the ensemble, the approach effectively captures the energetics of over 1060 different microscopic states. The COREX program used in the present invention is described, for example in U.S. Application Publication No. US20030032065A1 and U.S. application Ser No. 10/096,178, which are hereby incorporated by reference herein in their entirety.

A. Generation of Partly Folded States by COREX Algorithm

The partition function

$$( Q = {\sum }_{i = 0}^{N} exp ( - Δ {G}_{i} / RT ) )$$

is a sum over all states of the protein. This is an astronomical number even for a small protein. For example, if each amino acid of a 100 residue protein had ten possible conformations, the total number of states would be 10¹⁰⁰, which is a computationally intractable number. Fortunately, protein folding is a highly cooperative process and most states have almost zero probability and do not contribute to the partition function. This fact permits a significant simplification of the enumeration problem. It is desirable to develop a set of selection rules that will allow the creation of a subset that only contains the states that contribute to the partition function.

An approach to generating an ensemble of intermediate states for a particular protein is to use the high-resolution structure of the native state as a template and in a systematic way use the computer to unfold predetermined regions of the molecule in all possible combinations. The resolution of the results depends on the size and number of the regions (called folding units) used to generate the partially folded states. There are two basic assumptions in this algorithm: (1) the folded regions in partially folded states are native-like; and (2) the unfolded regions are assumed to be devoid of structure.

The COREX algorithm employs a block of windows of N_{w} amino acid residues each, which is used to partition the protein into different folding units. Each protein partition consists of Nᵤ folding units, where Nᵤ is equal to the ceiling of Nᵣₑₛ/N_{w}. Nᵣₑₛ is the total number of residues and N_{w} is the number of amino acid residues per window. The first partitioning of the protein is defined by moving the block of windows over the entire sequence of the protein beginning with the first residue. If Nᵣₑₛ/N_{w} is not an integer, the number of residues in the last unit is set equal to the remainder. This partitioning results in (2^{Nu,i}−2) partially folded intermediates generated by folding and unfolding the units in all possible combinations. Once the first partitioning is performed, a second partitioning is defined by sliding the block of windows one amino acid residue in the sequence. This process is continued until the entire sequence has been exhausted. The total number of distinct states generated by using this procedure is equal to 2+Σ(2^{Nu,i}−2), where the sum runs over all partitions and N_{U,i} is the number of folding units in partition i.

B. Calculation of Gibbs Energies

The free energy of each conformational state is calculated using the empirical parameterization of the free energy of each of the conformation states in the ensemble (D'Aquino et al., 1996; Gomez & Freire, 1995; Gomez et al., 1995; Xie & Freire, 1994a,b). This procedure involves calculation of the relative heat capacity (ΔCₚ), enthalpy (ΔH) and entropy (ΔS) of each state at the desired temperature.

Conformational entropies are evaluated by explicitly considering the following three contributions for each amino acid: (1) ΔS_{bu→ex}, the entropy change associated with the transfer of a side-chain that is buried in the interior of the protein to its surface; (2) ΔS_{ex→u}, the entropy change gained by a surface-exposed side-chain when the peptide backbone unfolds; and (3) ΔS_{bb}, the entropy change gained by the backbone itself upon unfolding. The magnitude of these terms for each amino acid residue is estimated by computational analysis of the probability of different conformers as a function of the dihedral and torsional angles (D'Aquino et al., 1996; Lee et al., 1994). Additional entropic contributions due to the presence of disulfide bridges were estimated as described by Pace et al. (1988). On average, these contributions account for about 95% of the entropy change for complete unfolding of the protein. The remaining unaccounted contributions (primarily protonation effects) are estimated from the difference between predicted and experimental Gibbs energies for complete unfolding under the specific experimental conditions and distributed evenly among all residues.

C. Hydrogen Exchange Protection Factors

Under experimental conditions in which the so-called EX2 regime is obeyed, the equilibrium constant for the following reaction is measured by hydrogen exchange experiments (e.g., see Bai et al., 1995):

$${NH}_{j} ( closed ) {⟷}^{{K}_{op , j}} {NH}_{j} ( open ) .$$

According to this reaction, Kₒₚⱼ is equal to the ratio between the sum of the concentrations of all conformations in which residue j is open and therefore able to exchange protons with the solvent, and the sum of the concentrations of all conformations in which residue j is closed. The standard interpretation is that slowly exchanging protons exchange with the solvent only after becoming exposed to it as a result of local, partial or global unfolding. The residues that are unfolded in partially folded states are not the only residues that become exposed to the solvent. Also the residues located in the so-called complementary regions become exposed to the solvent, i.e., the residues located in portions of the protein that remain folded but were structurally complementary to the regions of the protein that became unfolded (Freire et al., 1993). The commonly reported hydrogen exchange protection factors, PFⱼ, are equal to the inverse of the Kₒₚⱼ constants.

While the residue stability constants are purely thermodynamic quantities defined for all residues, the protection factors also contain non-thermodynamic contributions and are defined only for a subset of residues. Proline residues lack exchangeable amide protons and are not included. Residues with solvent-exposed amide groups in the native state are excluded (Pedersen et al., 1991). From a statistical standpoint, the protection factor for any given residue j can be defined as the ratio of the sum of the probabilities of the states in which residue j is closed, to the sum of the probabilities of the states in which residue j is open:

$${PF}_{j} = \frac{{\sum }_{( stateswithresiduejclosed )}^{} {P}_{i}}{{\sum }_{( stateswithresiduejopen )}^{} {P}_{i}} = \frac{{P}_{closed , j}}{{P}_{open , j}}$$

The statistical definition of the protection factors has the same form as that of the stability constants and can be expressed in terms of the folding probabilities as follows:

$${PF}_{j} = \frac{{P}_{f , j} - {P}_{f , xc , j}}{{P}_{nf , j} + {P}_{f , xc , j}}$$

the correction term P_{f,xcj} is the sum of the probabilities of all states in which residue j is folded, yet exchange competent. It is evident that the hydrogen exchange protection factors PFⱼ are equal to the stability constants per residue, κ_{fj} only when the P_{f,xc,j} terms are small. The most common situations in which a residue is folded but exposed to the solvent occurs when: (1) the amide group of the residue is exposed in the native state; and (2) the amide group of the residue becomes exposed due to its location in a region of the protein that is structurally complementary to an unfolded region.

It is clear from the above treatment that PFⱼ as well as κ_{fj} are statistically defined quantities pertaining to an ensemble of conformations rather than to a chemical reaction between two discrete states.

III. Structural Distribution of Cooperative Interactions in Proteins

Cooperative interactions link the behavior of different amino acid residues within a protein molecule. As a result, the effects of chemical or physical perturbations to any given residue are propagated to other residues by an intricate network of interactions. Very often, amino acids “sense” the effects of perturbations occurring at very distant locations in the protein molecule. Cooperative interactions are not intrinsically bi-directional and different residues play different roles within the intricate network of interactions existing in a protein. The effect of a perturbation to residue j on residue k is not necessarily equal to the effect of the same perturbation to residue k on residue j. Thus, the present invention utilizes COREX to map the network of cooperative interactions within a protein (Pan et al., 2000)

A. Residue-Specific Connectivities.

Residue-specific connectivity (RSC) is used to described the coupling between two residues. The correlation function is defined as:

$$RSC ( j , k ) = \frac{⟨ ( {S}_{j} - {\overline{S}}_{j} ) \cdot ( {S}_{k} - {\overline{S}}_{k} ) ⟩}{{( ⟨ {( {S}_{j} - {\overline{S}}_{j} )}^{2} ⟩ \cdot ⟨ {( {S}_{k} - {\overline{S}}_{k} )}^{2} ⟩ )}^{1 / {2}^{′}}}$$

where Sⱼ and Sₖ denote the folding state of residue j and k (if a residue is folded in a particular state, S=1; if the residue is unfolded, S=−1), and <Sⱼ> and <Sₖ> denote the average folding state of residues j and k over the ensemble. A positive value of the RSC indicates that a stabilization of residue j or k results in a stabilization of residue k or j (i.e., they display positive cooperativity), whereas a negative value indicates a stabilization of residue j or k leads to a destabilization of residue k or j (i.e., they display negative cooperativity). A value of zero means there is no correlation, and the residues are not energetically coupled.

Because RSC provides the mutual susceptibility of each residue to perturbations at every other residue, it can be used to probe the thermodynamic domain structure in proteins (Hilser et al., 1998; Pan et al., 2000). Yet further, in addition to providing thermodynamic domain assignments, the RSCs can also be used to explore how mutational effects propagate from their point of origin. Thus, it is envisioned that RSCs can determine how SNPs propagate from their point of origin and determine their relevance to the active site of the protein.

B. Functional Connectivities.

In addition to the pairwise correlations described by the RSCs, it is necessary to identify those joint correlations between the binding site as a whole and the rest of the protein. Accordingly, further embodiments of the present invention, define the functional connectivity (FC) as the connectivity between the entire binding site for a given ligand, x, and every residue in the protein:

FCₓ(j*, k*)=RSC(j, k)

where j* and k* are defined in one of two ways. For all residues, j and k, not involved in the binding pocket for ligand x, Sⱼ*=Sⱼ and Sₖ*=Sₖ in the RSC equation. For residues in the binding site for ligand x, however, Sⱼ* and/or Sₖ* are the average folding states over all of the nx residues in the binding site for ligand x:

$${Sj}^{*} = \frac{{\sum }_{y = 1}^{nx} Sj}{{n}_{x}}$$

The difference between RSCs and FCs is noteworthy. RSCs are determined by correlating the probabilities of states in which two particular residues are folded, independent of the folded state of other residues. FCs, on the other hand, correlate the probability of a residue being folded with the probability of a group of residues being folded. As such, FCs effectively amplify connectivity information that is often not seen in an analysis of the RSCs.

IV. MPMOD

MPMOD utilizes combinations of random searches of conformational space in the allowed regions of Ramachandran plot. The use of these random searches of conformational space provides a simple and useful tool to study the behavior of mini cyclic peptides. This is done using the simple hard sphere model to generate the stereochemically acceptable conformers and flexible disulfide bond modeling. The “rate” for SS bond loop closure as defined by N_{c}/Nₒ (where N_{c} is the number of conformers that can potentially form disulfide bond and Nₒ is the number of conformers that can not form a disulfide bond but have passed van der Waals check) becomes saturated when the ensemble has more than 1000 conformer. For the CXC and CXXC series of peptides, the modeled probability of loop closure behaves the same way as the experimentally determined equilibrium constant K_{c} for all the four types of the mini peptides. Both compare well after a common scale factor is applied. van der Waals interactions play a dominant role in loop closure for the small peptides CXC and CXXC.

MPMOD is an efficient method to generate disulfide bonded conformers. It takes about 10-20 CPU minutes to obtain 4000 disulfide bonded conformers CXXC using a Linux system on a Pentium III 450. Because the conformer CXC has higher probability of collision, it takes about 3 times more CPU time than to generate the CXXC. However, the consumed CPU time strongly depended on the criteria used to generate the conformer. The MPMOD program used in the present invention is described, for example in U.S. Application Publication No. US20030032065A1 and U.S. application Ser. No. 10/096,178, which are hereby incorporated by reference herein in their entirety.

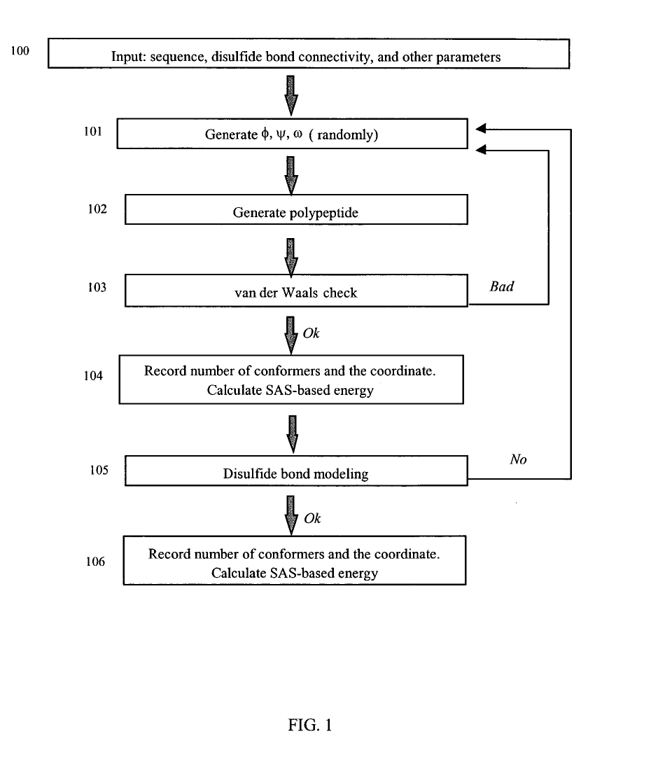

The flow chart illustrated in FIG. 1 shows the general program for MPMOD. The input parameters (step 101), such as the peptide sequence and disulfide bond connectivity, are loaded, then the conformational angles (φ, ψ, ω) are generated in the four maps (step 102). The atoms of main chain and side chain are generated base on the angles. The van der Waals checks are performed separately for the backbone atoms and side chain atoms (step 103). If there is a van der Waals violation, the conformer will be rejected. It will go back to get another set of conformational angles until the peptide is finished without any atom collisions. Then the coordinates of peptide are recorded and the solvent accessible surface (SAS) based energy is calculated (step 104). The disulfide bond is modeled to see if there is a disulfide bond is possible for the two residue pairs (step 105). If a disulfide bond is possible, the SAS energy for this conformer is calculated. If a disulfide bond is not possible, another set of conformational angles is tried and the procedure is repeated until a conformer with a disulfide bond is obtained. Finally, the SAS energy is calculated for this conformer again (step 106).

V. BEST/MPMOD to Determine Relevant SNPs

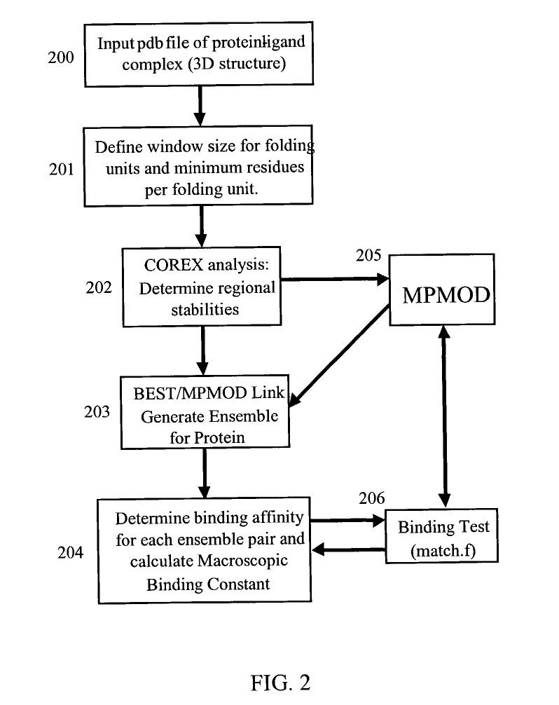

As used herein, “BEST” refers to a technology that models states in a combined fashion. For a given state, regions that are folded are modeled according to the high-resolution structure, while regions that are unfolded are modeled as having a conformational entropy (as opposed to being modeled as a large number of microscopic conformations). More specifically, COREX and BEST are used to determine thermodynamic space. For example, as the entropy change (ΔS) is related to the difference in the number of conformational states in the particular macroscopic state relative to the degrees of freedom in the reference of fully folded state, a large number of microscopic states can be represented by a single macroscopic state. The implication of this dual modeling procedure is that a macroscopic state that has 10 residues unfolded (with a minimum of 4 conformations per residue) would require modeling over 1 million explicit microscopic conformational states. The end result of the calculation is that with 100,000 macroscopic states in the ensemble, the present invention effectively captures the energetics of over 10⁶⁰ different microscopic states. The mini-protein modeling of disulfides (MPMOD) portion of the present invention explicitly models conformations for small regions of the protein that are shown to be unstable by BEST, thus, MPMOD is used to determine structural space or detail at the molecular level. Once the unstable regions are identified using BEST, the two flanking residues of the unstable region are anchored by fixing the residues in the conformations found in the high-resolution structure. Conformations are generated by projecting the loop as described in MPMOD. The combination of the BEST and MPMOD programs, termed BEST/MPMOD, allows the explicit modeling of conformations for proteins of any size. Thus, as shown in FIG. 2, step 200 includes inputting a three-dimensional structure of a protein-ligand complex. The structure can be inputted directly or it may be obtained for any database well known and used by those of skill in the art. Next, step 201 involves defining windows size for folding units and minimum residues per folding unit. Step 202 comprises performing COREX analysis to determine regional stabilities. Step 203 is linked to step 205 which is MPMOD which is linked to step 203, the BEST/MPMOD module to generate an ensemble for the protein. Next, step 204 includes determining binding affinity for each ensemble pair and calculating macroscopic binding constant, which is linked to step 206, which is a binding test module. The binding test module is linked to MPMOD, or step 205. The BEST/MPMOD programs used in the present invention are described, for example in U.S. Application Publication No. US20030032065A1 and U.S. application Ser. No. 10/096,178, which are hereby incorporated by reference herein in their entirety

The computer assisted programs of the present invention can be combined with a variety of databases that would eliminate guess work or decrease the amount of time that is necessary for the programs to run. One such database that may be used is a database that defines thermodynamic propensities of amino acids (See, i.e., U.S. Application Publication No. US20020193566A1, which is incorporated in its entirety by reference).

Yet further, specific embodiments of the present invention provide that the database comprises nonhomologus proteins. Nonhomologus proteins can be obtained from any of the protein databases, for example, but not limited to the Protein Data Bank. One of skill in the art is cognizant the any database that contains SNP related data may be used in the present invention. In fact, these databases are used in the computer modeling programs in the present invention to develop a database that contains SNPs that are relevant to the function of the protein.

In specific embodiments, the computational methods of the present invention determine the energetic connectivities between different structural elements by determining the residue-specific connectivities and the functional connectivities. This information is used to develop a database that contains SNPs that are relevant to the function of the protein.

The database of the present invention can then be used to predict a subset of the population at risk for an adverse side effect of a pharmaceutical composition. Yet further, the database may be used to target a pharmaceutical composition for an active site of a protein. Specifically, the database is used to determine the interactions of SNPs on the active site of a protein and design the pharmaceutical composition to overcome the interactions of the SNP so that the pharmaceutical composition binds to the active site of the protein.

Yet further, the present invention comprises a method of developing a protein database comprising inputting a high resolution structure of a protein; generating an ensemble of conformational states; determining the probability of each conformation state; calculating the residue-specific connectivity and function connectivity of each conformation state.

The generating of an ensemble of incrementally different conformations by combinatorial unfolding of a set of predefined folding units in all possible combinations comprises dividing the protein into folding units by placing a block of windows over the entire sequence of the protein and sliding the block of windows one residue at a time.

The determining the probability of each conformational state comprises determining the free energy (Gᵢ) of each of the conformational states in the ensemble; determining the Boltzmann weight [Kᵢ=exp(−Gᵢ/RT)] of each state; and determining the probability of each state using the equation

$${P}_{i} = \frac{{K}_{i}}{\sum {K}_{i}^{′}} .$$

Once thermodynamic space has been determined, it is contemplated that MPMOD can be used to determine structural space. The all-atom computational approach of MPMOD comprises: searching of conformational space in the allowed regions of the Ramachandran plots; eliminating grossly improbable conformers by a hard sphere approximation; searching flexible disulfide bond models; and calculating a solvent accessible surface (SAS) based energy. The method may further comprise calculating the probability of the ensemble forming a disulfide.

In further embodiments, the BEST/MPMOD method is used to design a protein pharmaceutical exhibiting optimized pharmaceutical properties, i.e., increased binding affinity. The method comprises the steps of: obtaining a test data set of variants of the protein pharmaceutical, wherein the variants comprise single nucleotide polymorphisms; preparing a library of ensemble derived properties for the test data set using a computer based method; obtaining experimental data for a given property for each protein variant within the test data set; deriving a parametric equation using the experimental data and the library of ensemble derived properties; and creating a protein pharmaceutical using the information obtained by the above steps to provide optimized pharmaceutical properties. In specific embodiments, the optimized pharmaceutical property is binding affinity.

Binding affinity is the measure of the overall free energy of the interaction between the protein and the ligand. The magnitude of the affinity determines whether a particular interaction is relevant under a given set of conditions. Whether or not any particular affinity of a protein for a ligand is significant depends on the concentration of the ligand present for the protein to encounter. Assays for determining binding affinity include, but are not limited to, surface plasmon resonance, Western blot, ELISA, DNase footprinting, and gel mobility shift assays. The ligand may be protein or non-protein. The ligand may be, but is not limited to, a receptor, a coenzyme, or a non-proteinaceous chemical compound. Binding affinity between a protein and ligand may be measured by the association or dissociation constant of the binding between the protein and the ligand. Entropy of binding between the protein and ligand may be decreased by stabilizing structures similar to that of the protein in a bound state with the ligand. van der Waals calculations can be performed with the protein and the ligand to determine whether binding conformation will be sterically allowed.

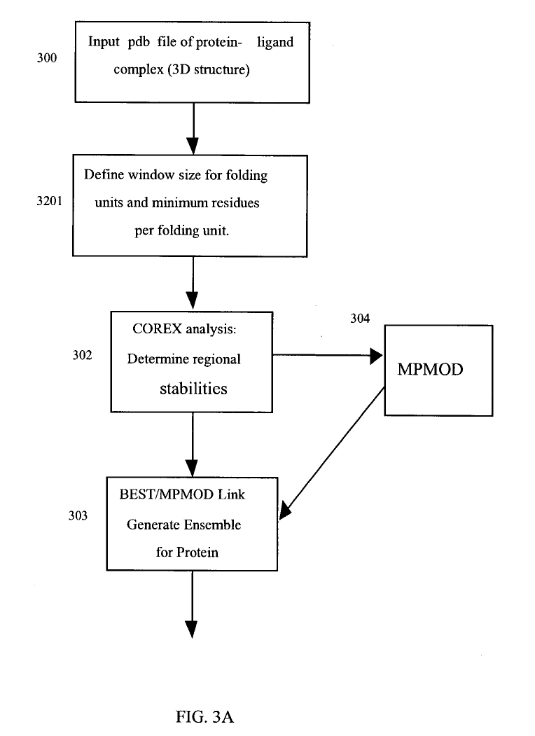

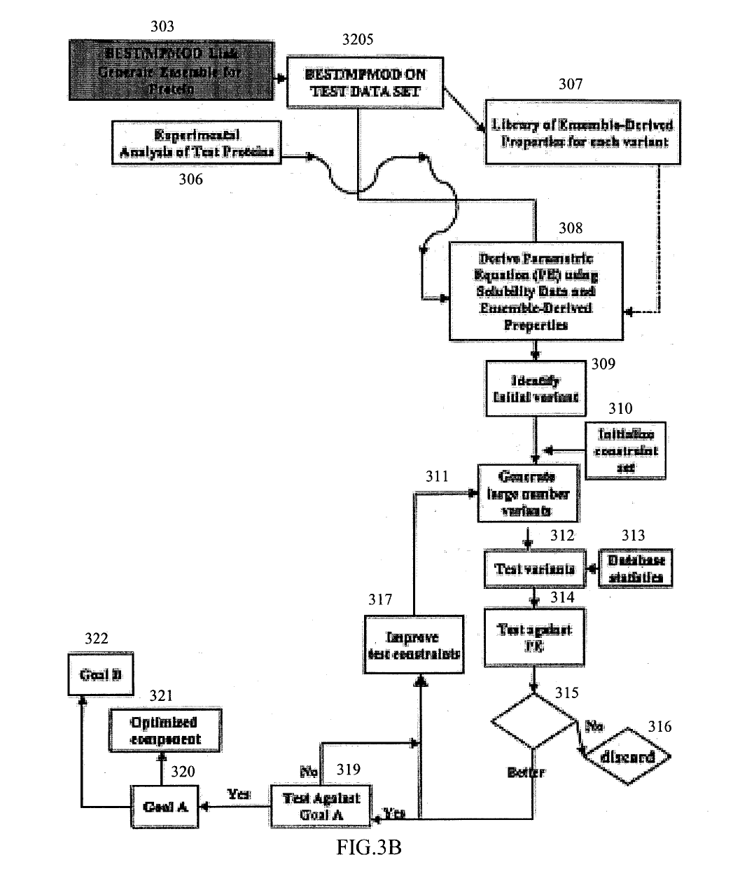

In a specific example, the BEST/MPMOD method of designing a protein pharmaceutical to exhibit optimized pharmaceutical properties is shown in FIGS. 3A and 3B. Step 300 is inputting a high resolution structure of said protein pharmaceutical into a computer-assisted modeling program. Step 301 comprises defining a window size for folding units. COREX analysis is performed in step 302 and MPMOD is performed in step 304. The combination of these programs results in the generation of a test data set in step 305. The test data contains variants comprising SNPs. Data are obtained by the following steps: obtaining an ensemble of incrementally different conformations of said protein by combinatorial unfolding of a set of predefined folding units; determining a probability of each conformational state of said protein pharmaceutical; calculating protection factors of residues within the protein; determining energetic connectivities between different structural elements of said protein; identifying regions of said protein that are unstable; obtaining ensembles of conformers of the unstable region using an all-atom computational approach facilitated by random selection of phi/psi and torsional angles allowing deviation of the geometrical parameters from mean values; and determining a fraction of conformations of the protein exhibiting optimized pharmaceutical properties. In step 307, the step comprises mutating the amino acid sequence of the protein pharmaceutical to provide a variant and repeating repeating the steps from determining pharmaceutical properties through determining a fraction of conformations a given number of cycles in order to prepare a library of ensemble-derived properties for each variant. Step 308 is deriving a parametric equation using the pharmaceutical properties of each variant and the library of ensemble-derived properties. Next, an initial lead variant is identified in step 309. In step 310, a constraint set is initialized. Based on step 309, a large number of variants is obtained in step 311. Next, in step 32, variants are tested to select a lead SNP set based on a database. Step 314, is testing the lead in the parametric equation. Next, step 315 is to determine if the lead has optimized pharmaceutical delivery properties over the prior lead tested. In step 319, the variant is compared to the goal for each property, Goal A (220) or Goal B (222). Step 321, is to create the variant of the protein pharmaceutical with the structural characteristics found by the above steps to provide optimized pharmaceutical delivery properties. Optionally, the steps from obtaining a large number of variants through determining if the lead is optimized can be repeated to sufficiently optimize the pharmaceutical properties of the protein for its intended pharmaceutical use. Pharmaceutical properties can include but are not limited to, increased binding affinity. One of skill in the art realizes that the BEST/MPMOD method of protein design is not limited to protein pharmaceuticals.

Any type of logic may be used to determine which mutations to make to create the data set. Types of logic include but are not limited to any Monte Carlo weighted selection procedure and neural networks.

In this invention, any type of commercially available data analysis may be used to get the relationship between the calculated and experimental data. An overdetermined data set is required to perform the invention. An arbitrarily set, protein dependent, number of mutations are examined for any pharmaceutical properties of interest to provide a data set and a “jackknife” analysis if performed on the data set. In the “jackknife analysis”, a part of the data set is removed and the data reanalyzed. If the analysis is not statistically different, then the data set is overdetermined. Any other statistical method of testing whether the data set is overdetermined may be used in this invention. Examples of statistical methods that may be used for analyzing the data are principle component analysis and singular value decomposition. It is possible that the data set for one pharmaceutical property may be overdetermined while the data set for another pharmaceutical property is underdetermined. If a data set is underdetermined, it is necessary to add more mutation variants to the data set.

VI. Mutagenesis

Where employed, mutagenesis is accomplished by a variety of standard, mutagenic procedures. Mutation is the process whereby changes occur in the quantity or structure of an organism. Changes may be the consequence of point mutations that involve the removal, addition or substitution of a single nucleotide base within a DNA sequence, or they may be the consequence of changes involving the insertion or deletion of large numbers of nucleotides.

Structure-guided site-specific mutagenesis represents a powerful tool for the dissection and engineering of protein interactions (Wells, 1996). The technique provides for the preparation and testing of sequence variants by introducing one or more nucleotide sequence changes into a selected DNA.

Site-specific mutagenesis uses specific oligonucleotide sequences which encode the DNA sequence of the desired mutation, as well as a sufficient number of adjacent, unmodified nucleotides. In this way, a primer sequence is provided with sufficient size and complexity to form a stable duplex on both sides of the deletion junction being traversed. A primer of about 17 to 25 nucleotides in length is preferred, with about 5 to 10 residues on both sides of the junction of the sequence being altered.

The technique typically employs a bacteriophage vector that exists in both a single-stranded and double-stranded form. Vectors useful in site-directed mutagenesis include vectors such as the M13 phage. These phage vectors are commercially available and their use is generally well known to those skilled in the art. Double-stranded plasmids are also routinely employed in site-directed mutagenesis, which eliminates the step of transferring the gene of interest from a phage to a plasmid.

In general, one first obtains a single-stranded vector, or melts two strands of a double-stranded vector, which includes within its sequence a DNA sequence encoding the desired protein or genetic element. An oligonucleotide primer bearing the desired mutated sequence, synthetically prepared, is then annealed with the single-stranded DNA preparation, taking into account the degree of mismatch when selecting hybridization conditions. The hybridized product is subjected to DNA polymerizing enzymes such as E. coli polymerase I (Klenow fragment) in order to complete the synthesis of the mutation-bearing strand. Thus, a heteroduplex is formed, wherein one strand encodes the original non-mutated sequence, and the second strand bears the desired mutation. This heteroduplex vector is then used to transform appropriate host cells, such as E. coli cells, and clones are selected that include recombinant vectors bearing the mutated sequence arrangement.

Other methods of site-directed mutagenesis are disclosed in U.S. Pat. Nos. 5,220,007; 5,284,760; 5,354,670; 5,366,878; 5,389,514; 5,635,377; and 5,789,166.

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example

### Use of COREX, BEST and MPMOD to Identify Relevant SNPs

To describe the use of COREX, BEST and MPMOD for identifying relevant SNPs, calculations for Human multiple tumor suppressor protein Cyclin-dependent kinase 4 inhibitor A (Protein name: CDK4I Gene Name: CDKN2A, SWISSPROT entry: CDN2_HUMAN) have been preformed.

CDK4I acts as a negative regulator of cell proliferation. It interacts with cylcin-dependent kinase 4 and 6 that are essential for cell division and prevent them from interacting with Cyclin D. CDKN2A codes for 156 residue protein of molecular weight 16.5 KDa. CDK4I is an alpha+beta structure and belongs to the family of Ankyrin repeat proteins (PDB code: 1B17, 1A5E, 1DC2). Several point mutations associated with this protein results in aberrant phenotypes like melanoma, Li-Fraumeni syndrome, neural, pancreatic, lung and esophagal tumors.

Briefly, the X-ray crystallography experimental structure of CDK4I (PDB code 1B17) complexed with Cyclin-dependent kinase 6 (CDK6) was downloaded from the Protein Data Bank. A list of 29 missense and 3 nonsense nucleotide substitution mutations in the CDKN2A gene was noted from the Human Gene Mutation Database. The NMR structure of CDK4I was used to generate an ensemble of states by defining a fold window size of 8 residues. COREX was used for analyzing regional or residue specific stabilities expressed as LnKf.

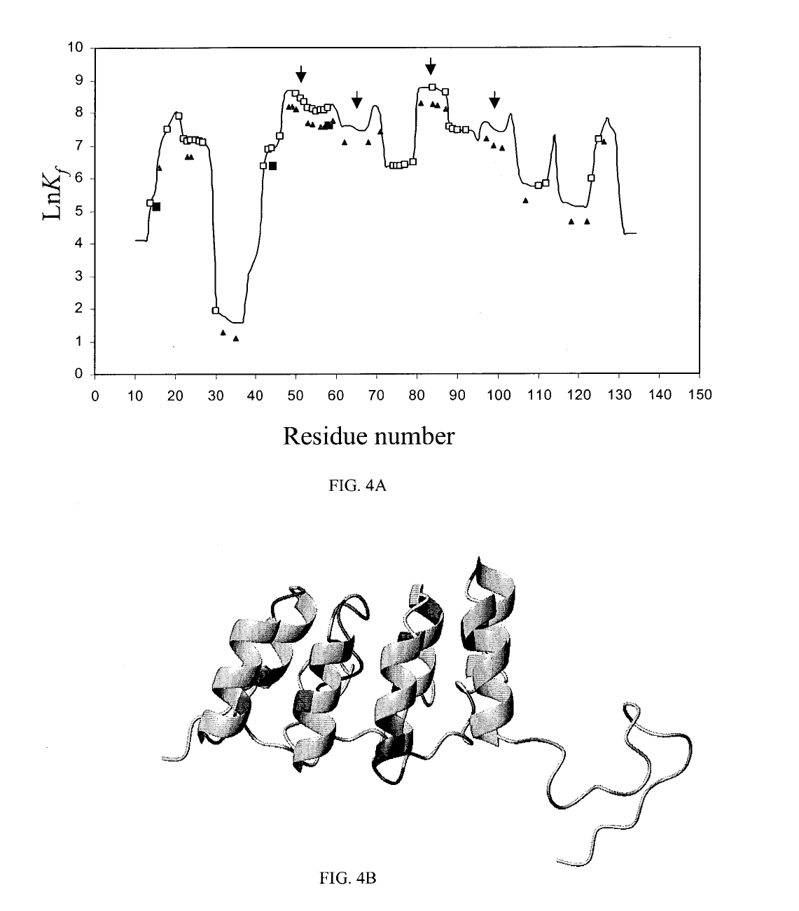

FIG. 4A is the thermodynamic stability plot calculated for individual residues of CDK4I using COREX with entropy weighting factor of 1.049 at a temperature of 25° C. High stability regions were defined by residues that occured with a stability factor LnKf greater than 6.0. A total of 86 residues were found in the high stability region that included 77% of the 31 observed SNPs and 85% of 39 residues found to make contact with CDK6. Only 10 SNPs were found among 39 residues that made contact (32%) whereas 77% of observed disease causing SNPs occurred in the high stability region. Thus, at the first-step of ensemble based calculation using COREX, the inventors were able to obtain a 2.5 time higher prediction of disease causing SNPs compared to a simple prediction based on protein-protein contacts alone. Mutations that resulted in aberrant phenotype were found clustered in the high stability region (indicated by a vertical arrow in FIG. 4A).

### REFERENCES

All patents and publications mentioned in the specification are indicative of the level of those skilled in the art to which the invention pertains. All patents and publications are herein incorporated by reference to the same extent as if each individual publication was specifically and individually indicated to be incorporated by reference.

U.S. Pat. No. 4,554,101

U.S. Pat. No. 5,220,007

U.S. Pat. No. 5,284,760

U.S. Pat. No. 5,354,670

U.S. Pat. No. 5,366,878

U.S. Pat. No. 5,389,514

U.S. Pat. No. 5,440,013

U.S. Pat. No. 5,446,128

U.S. Pat. No. 5,475,085

U.S. Pat. No. 5,618,914

U.S. Pat. No. 5,635,377

U.S. Pat. No. 5,670,155

U.S. Pat. No. 5,672,681

U.S. Pat. No. 5,674,976

U.S. Pat. No. 5,789,166.

U.S. Pat. No. 5,929,237

Akke, M., et al., (1993) Biochemistry 32, 9832-9844.

Alberts et al. (1994) Molecular Biology of the Cell, p 57.

Bai, Y. et al., (1995). Science, 269, 192-197.

Baldwin, R. L. (1986). Proc. Natl. Acad. Sci. USA, 83, 8069-8072.

Bolin, J. T., et al., (1982) J. Biol. Chem. 257, 13650-13662.

Bruccoleri, R. E. and Karplus, M. (1987), Biopolymers, Vol.26, 137-168.

Bystroff, C. & Kraut, J. (1991) Biochemistry 30, 2227-2239.

Bystroff, C., Oatley, S. J. & Kraut, J. (1990) Biochemistry 29, 3263-3277.

Cameron, C. E. & Benkovic, S. J. Biochemistry 36, 15792-15800.

D'Aquino, J. A., et al., (1996). Proteins: Struct. Funct. Genet. 25, 143-156.

Fierke, C. A. & Benkovic, S. J. (1989) Biochemistry 28, 478-486.

Fierke, C. A., et al., (1987) Biochemistry 26, 4085-4092.

Freire E. & Xie, D. (1994). Biophys. Chem. 51, 243-251.

Freire, E. (1995). Annu. Rev. Biophys. Biomol. Struct. 24, 141-165.

Freire, E. (1999) Proc. Natl. Acad. Sci. USA 96, 10118-10122.

Freire, E. et al., (1993). Proteins: Struct. Funct. Genet. 17, 111-123.

Gomez J. & Friere, E. (1995). J. Mol. Biol. 252, 337-350.

Gomez, J., et al., (1995). Proteins: Struct. Funct. Genet. 22, 404-412.

Griko, Y., et al., (1994). Biochemistry, 33, 1889-1899.

Griko, Y. V., et al., (1995). J. Mol. Biol. 252, 447-459.

Haynie, D. T. & Freire, E. (1993). Proteins: Struct. Funct. Genet. 16, 115-140.

Hilser, V. J. & Freire, E. (1996) J. Mol. Biol. 262, 756-772.

Hilser, V. J. & Freire, E. (1997) Proteins Struct. Funct. Genet. 27, 171-183.

Hilser, V. J., et al., (1998) Proc. Natl. Acad. Sci. USA 95, 9903-9908.

Hilser, V. J., et al., (1996) Proteins 26, 123-133.

Hilser, V. J., et al., (1997) Biophys. Chem. 64, 69-79.

Iijima, H., et al., (1987) Prot: Struct, Funct, and Genet 2, 330-339

Jacobs, M. D. & Fox, R. O. (1994). Proc. Natl Acad. Sci. USA, 91, 449-453.

Jennings, P. A. & Wright, P. E. (1993). Science, 262, 892-896.

Johannesson et al., (1999) J. Med. Chem. 42:601-608.

Johnson, M. S. et al., (1993) J. Mol. Biol., 231:735-52.

Jones, B. E. & Matthews, C. R. (1995). Protein Sci. 4, 167-177.

Kyte & Doolittle (1982) J Mol Biol. 157:105-32.

Lee, K. H. et al., (1994). Proteins: Struct. Funct. Genet. 20, 68-84.

Matthews, C. R. (1993). Annu. Rev. Biochem. 62, 653-683.

Metropolis, N., et al.,(1953) J. Chem. Phys. 21, 1087-1092.

Momany, F. A., et al., (1975) J. Phys. Chem. 79, 2361-2381.

Murphy, K. P. & Freire, E. (1992) Adv. Protein Chem. 43, 313-361.

Murphy, K. P., et al., (1992). J. Mol. Biol. 227, 293-306.

Ohmae, E., et al., (1996) J. Biochem. 119, 703-710.

Ohmae, E., et al., (1998) J. Biochem. 123, 839-846.

Olejniczak, E. T., et al., (1997) Biochemistry 36, 4118-4124.

Pabo, C. O. & Lewis, M. (1982) Nature (London) 298, 443-447.

Pace, C. N., et al., (1988). J. Biol. Chem. 263, 11820-11825.

Pedersen, T. G., et al., (1991). J. Mol. Biol. 218, 413-426.

Peng, Z. & Kim, P. S. (1994). Biochemistry, 33, 2136-2141.

Ponder, J. W. & Richards, F. M. (1987), J. Mol. Biol. Vol 193, 775-791

Ramachandran, et al., (1963). J. Mol. Biol. 7, 95.

Remington's Pharmaceutical Sciences, 18th Ed. Mack Printing Company, 1990.

Schulman, B. A., et al., (1995). J. Mol. Biol. 253, 651-657.

Sosnick, T. R., et al., (1994). Nature Struct. Biol. 1, 149-156.

Stivers, J. T., et al., Biochemistry 35, 16036-16047.

Vita et al., 1998, Biopolymers 47:93-100.

Warren, M. S., et al., (1991) Biochemistry 30, 11092-11103.

Weisshoff et al., 1999, Eur. J. Biochem. 259:776-788.

Wells, J. A. (1996) Proc Natl Acad Sci USA. 93(1):1-6.

Xie, D. & Freire, E. (1994a). Proteins: Struct. Funct. Genet. 19: 291-301.

Xie, D. & Freire, E. (1994b). J. Mol. Biol. 242: 62-80.

Xie, D., Fox, R. & Freire, E. (1994). Protein Sci. 3, 2175-2184

Yu, L., et al., (1996) Biochemistry 35, 9661-9666.

Zidek, L., et al., (1999) Nat. Struct. Biol. 6, 1118-1121.

Although the present invention and its advantages have been described in detail, it should be understood that various changes, substitutions and alterations can be made herein without departing from the spirit and scope of the invention as defined by the appended claims. Moreover, the scope of the present application is not intended to be limited to the particular embodiments of the process, machine, manufacture, composition of matter, means, methods and steps described in the specification. As one of ordinary skill in the art will readily appreciate from the disclosure of the present invention, processes, machines, manufacture, compositions of matter, means, methods, or steps, presently existing or later to be developed that perform substantially the same function or achieve substantially the same result as the corresponding embodiments described herein may be utilized according to the present invention. Accordingly, the appended claims are intended to include within their scope such processes, machines, manufacture, compositions of matter, means, methods, or steps.

What is claimed is:

1. A protein database comprising nonhomologus proteins having identified single nucleotide polymorphisms that are relevant to the function of the protein.

2. The database of claim 1, wherein the function is binding of the protein.

3. The database of claim 1, wherein the database is determined by a computational method comprising the step of determining the residue-specific connectivity between residues in the protein according to the equation
$$RSC ( j , k ) = \frac{⟨ ( {S}_{j} - {\overline{S}}_{j} ) \cdot ( {S}_{k} - {\overline{S}}_{k} ) ⟩}{{( ⟨ {( {S}_{j} - {\overline{S}}_{j} )}^{2} ⟩ \cdot ⟨ {( {S}_{k} - {\overline{S}}_{k} )}^{2} ⟩ )}^{1 / {2}^{′}}} .$$

4. The database of claim 1, wherein the database is determined by a computational method comprising the step of determining the functional connectivity between the binding site of the protein and every residue in the protein according to the equation
FCₓ(j*,k*)=RSC(j,k).

5. A method of predicting a subset of the population at risk for an adverse side effect of a pharmaceutical composition comprising using the database of claim 1 to determine the subset having a relevant single nucleotide polymorphism interfering with an active site of a protein.

6. A method of targeting a pharmaceutical composition for an active site of a protein comprising using the database of claim 1 to determine the interactions of single nucleotide polymorphisms on the active site of a protein and designing the pharmaceutical composition to overcome the interactions of the single nucleotide polymorphism so that the pharmaceutical composition binds to the active site of the protein.

7. A method of developing a protein database comprising the steps of:
inputting high resolution structures of proteins;
generating an ensemble of incrementally different conformational states by combinatorial unfolding of a set of predefined folding units in all possible combinations of each protein;
determining the probability of each said conformational state;
calculating a residue-specific connectivity of each said conformational state; and
calculating a functional connectivity of each of said conformation state.

8. A system for developing a protein database having identified single nucleotide polymorphisms that are relevant to the function of the protein comprising
a protein database having a data structure for protein data, said data structure including data fields for relevant single nucleotide polymorphisms; and
a computer-based program for identifying protein data for said database, said program having
an input module for receiving high resolution structure data for one or more proteins, and
a processing module for determining the relevance of single nucleotide polymorphisms in the active site of one or more proteins and storing said data into said data fields of said protein database.

9. The system of claim 8, wherein said computer program further includes a display module for producing one or more graphical reports to a screen or a print-out.

10. A database having a data structure which stores information defining relevant single nucleotide polymorphism groups, said database comprising:
a field for storing a value of an amino acid name or amino acid abbreviation; and
one or more classification fields for storing a value representing a numerical value for a relevant single nucleotide polymorphism.

11. A method of designing a protein pharmaceutical exhibiting optimized pharmaceutical properties comprising the steps of:
i. obtaining a test data set of variants of the protein pharmaceutical, wherein the variants comprise single nucleotide polymorphisms;
ii. preparing a library of ensemble derived properties for the test data set using a computer based method;
iii. obtaining experimental data for a given property for each protein variant within the test data set;
iv. deriving a parametric equation using the experimental data and the library of ensemble derived properties; and
v. creating a protein pharmaceutical using the information obtained by the above steps to provide optimized pharmaceutical properties.

12. The method of claim 11, wherein the optimized property is increased binding affinity.

13. A method of identifying relevant single nucleotide polymorphisms comprising the steps of:
inputting high resolution structures of proteins;
generating an ensemble of incrementally different conformational states by combinatorial unfolding of a set of predefined folding units in all possible combinations of each protein;
determining the probability of each said conformational state;
calculating a residue-specific connectivity of each said conformational state; and
calculating a functional connectivity of each of said conformation state.
